(19) United States
(12) Patent Application Publication
(10) Pub. No.: US 2012/0130377 A1
(43) Pub. Date: May 24, 2012

(54) Burr Hole Drill Alignment Guide

(76) Inventors: Donald Bobo, Fountain Valley, CA (US); David R. Asbury, Lake Elsinore, CA (US)

(21) Appl. No.: 13/145,539

(22) PCT Filed: Jan. 21, 2010
(86) PCT No.: PCT/US10/21674

§ 371 Date: Feb. 6, 2012

Related U.S. Application Data
(60) Provisional application No. 61/205,377, filed on Jan. 21, 2009.

(51) Int. Cl. A61B 17/17; A61B 17/16

(52) U.S. Cl. 606/80

(57) ABSTRACT

A burr hole drill guide preferably includes a guide body having a guide passage that allows a drill bit to enter. Once the user has aligned the drill bit to the desired orientation, the guide can be lifted upwards, allowing a gap to pass over an upper, narrow attachment region of the drill bit and moved elsewhere (i.e., away from the procedure). In this respect, the surgeon is aware of the desired drilling angle, but is less restricted and better able to view the drill during the drilling process.

### RELATED APPLICATIONS

This application claims priority to U.S. Provisional Application Ser. No. 61/205,377 filed Jan. 21, 2009 entitled Burr Hole Alignment Tripod, which is hereby incorporated herein by reference.

### BACKGROUND OF THE INVENTION

Ventricular catheters are placed in the ventricular system of the brain through a burr hole drilled in the skull. Typically, doctors reference “landmarks” on the skull of the patient to determine the appropriate location to drill. Additionally, the drill must be carefully angled relative to the patient's skull so that the drill enters into the ventricle. If this angle is incorrectly aligned, it may be difficult or even impossible to advance a ventricular catheter into the patient's ventricle.

Generally, a drill bit should be perpendicular relative to the surface of the patient's skull to properly align with a target ventricle. However, since the topography of human skulls is irregular, especially near burr hole target locations, proper, perpendicular alignment can be difficult for a doctor to achieve without guidance.

The prior art contains a number of burr hole drill guides, such as those found in U.S. Pat. Nos. 7,033,367; 6,206,885; 5,207,681; 4,970,926; 4,931,056; 4,821,716; and 4,613,324, the contents of which are hereby incorporated by reference. While these guides provide alignment for both a burr hole drill and a ventricular catheter, they are typically expensive for users and require extra time to properly position, configure and use. Hence, there is a need for an improved and inexpensive guide device for use in drilling a burr hole.

### SUMMARY OF THE INVENTION

A burr hole drill guide preferably includes a guide body having a guide passage that allows a drill bit to enter. Once the user has aligned the drill bit to the desired orientation, the guide can be lifted upwards, allowing a gap to pass over an upper, narrow attachment region of the drill bit and moved elsewhere (i.e., away from the procedure). In this respect, the surgeon is aware of the desired drilling angle, but is less restricted and better able to view the drill during the drilling process.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 illustrates a perspective view of a preferred embodiment of a burr hole drill guide according to the present invention;

FIG. 2 illustrates a front profile view of the guide of FIG. 1;

FIG. 3 illustrates a side profile view of the guide of FIG. 1;

FIG. 4 illustrates a cross sectional view taken along lines 4 in FIG. 2;

FIG. 5 illustrates a top cross sectional view of the guide from FIG. 1;

FIG. 6 illustrates a top view of the guide from FIG. 1; and,

FIG. 7 illustrates a perspective view of the guide from FIG. 1 and a drill bit.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

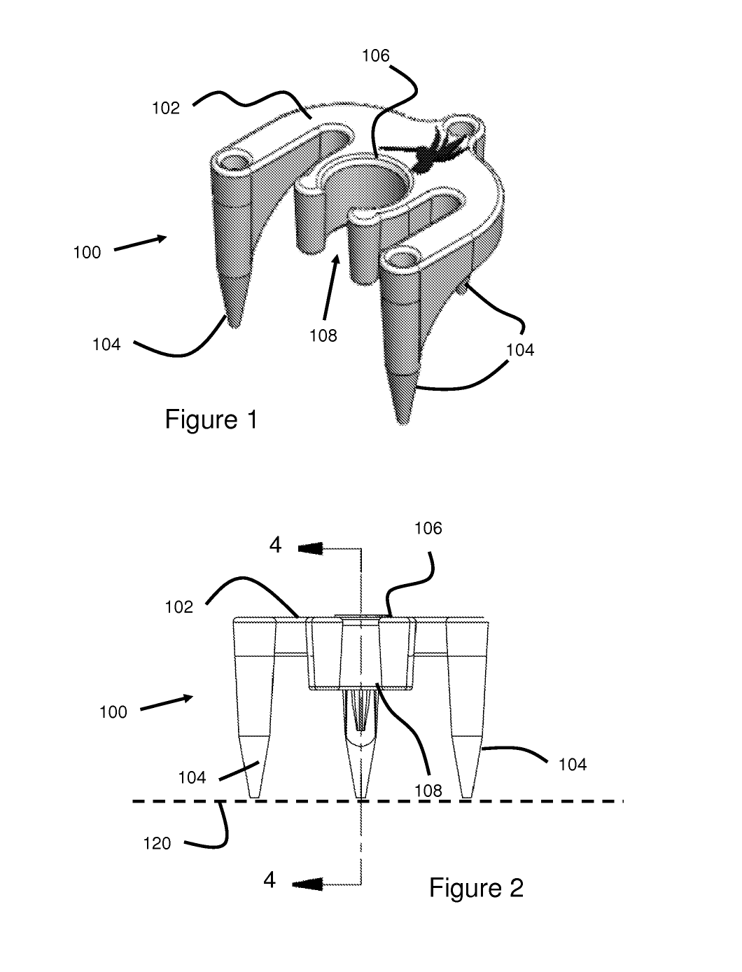

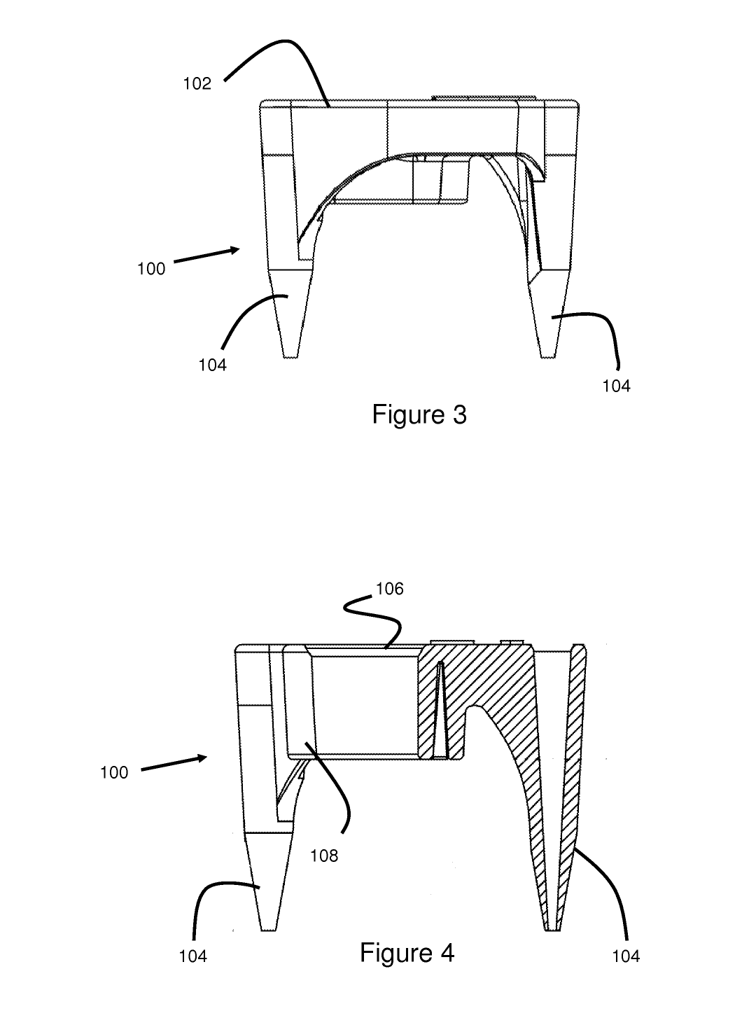

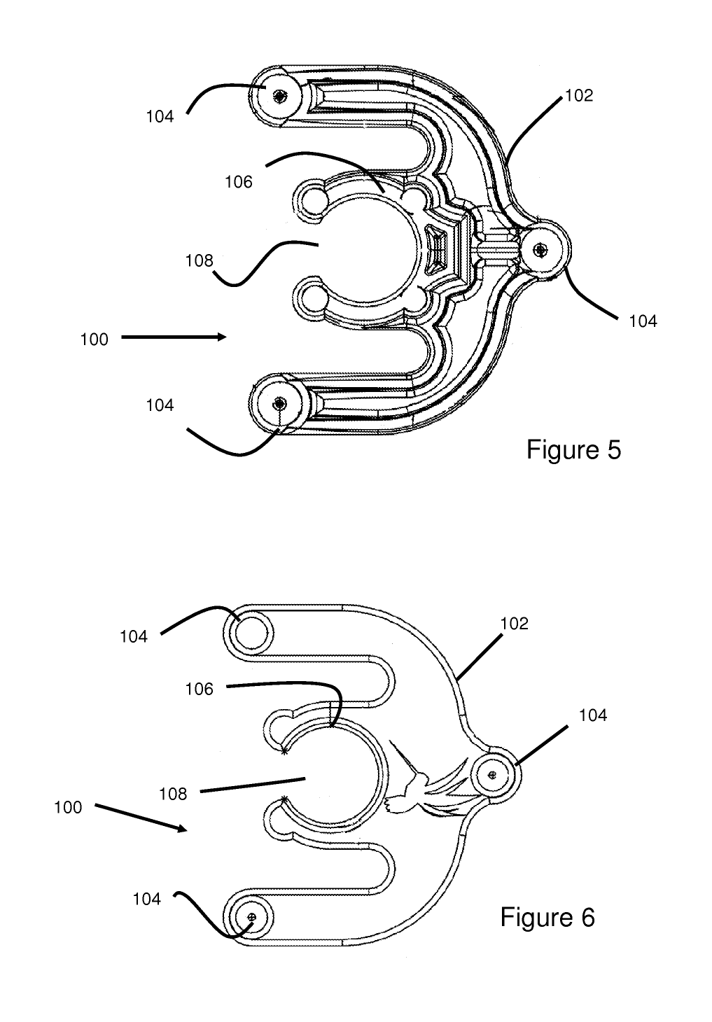

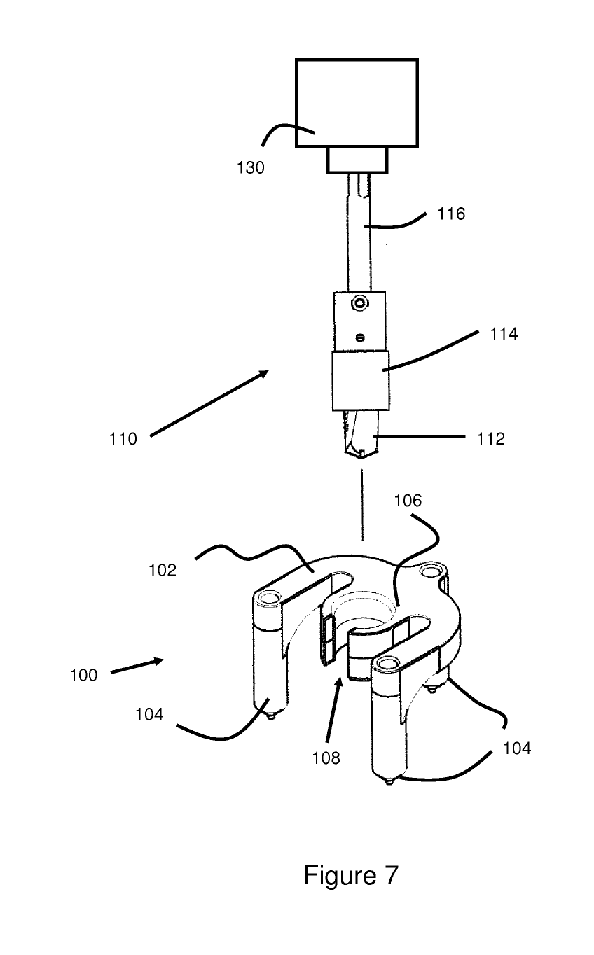

FIGS. 1-7 illustrate various views of a burr hole drill guide 100 according to a preferred embodiment of the present invention. Generally, the guide 100 includes a guide body 102 having a guide passage 106 that allows a drill bit 110 (seen in FIG. 7) to enter. Once the user has aligned the drill bit 110 to the desired orientation, the guide 100 can be lifted upwards, allowing a gap 108 to pass over an upper, narrow attachment region 116 of the drill bit 110 and moved elsewhere (i.e., away from the procedure). In this respect, the surgeon is aware of the desired drilling angle, but is less restricted and better able to view the drill during the drilling process.

The guide body 102 has a generally C-shaped body with a center region forming the guide passage 106. In the present example, the guide passage 106 is formed from two finger-like projections that create the gap 108 along a longitudinal area of the passage 106.

Preferably, the guide body 102 includes three leg members 104 that extend from its underside. Each leg member 104 has a generally tapered shape that terminates so as to provide traction when placed on the skull. For example, each leg 104 can have a point, screw, friction pad, or similar mechanism to retain position against skin. Preferably, each leg 104 is integral with the guide body 102, however, portions of the legs 104 may also be removable, replaceable or adjustable to modify the height of the guide 100.

Preferably, the guide body 102 and legs 104 are composed of a molded polymer. However, other rigid materials such as metal can also be used. Further, combinations of metal and polymer are also possible.

Turning to FIG. 7, a drill bit 110 includes the drill head 112, a stop member 114 and the narrow attachment region 116. The stop member 114 has an outer diameter that is slightly smaller than the interior diameter of the guide passage 106, allowing the drill head 112 and stop member 114 to pass through. For example, the interior diameter of the guide passage 106 can be about 0.473″ and the outer diameter of the stop member 114 can be about 0.471″. Assuming a rigid material calculation of angulation misalignment with guide 100 is tan (angle misalignment)*L=D1−D2 where D1 is the inner diameter of passage 106, D2 is outer diameter of the stop member 114, and L is the length of the passage 106 now set at 0.47″. This means that for a 1° angulation misalignment we can have a diameter difference of 0.008″. The alignment is held nearly net, by using contact ribs on the guide 100.

Preferably, the guide passage 106 is suspended above the skull (i.e., a virtual plane 120 seen in FIG. 2 that is formed between the three legs 104) such that when the drill bit 110 passes through the passage 106 and the drill head 112 touches the skull, a portion of the drill stop 114 remains in the passage 102. In this respect, the passage 106 forces the drill bit to maintain an orientation that is perpendicular to the virtual plane 120 formed between the three legs 104. For example, height or depth of the guide passage 106 can be about 0.47″, the guide passage 106 can be positioned over the skull (i.e., the virtual plane 120) at about 0.7″, and the stop 114 can be about 1.0″ in length.

As previously discussed, the drill head 112 and drill stop 114 have diameters larger than the gap 108 formed along the passage 106 and therefore cannot pass through the gap 108. A portion of the upper attachment region 116 is connected to a drill 130 and has a diameter that allows it to laterally pass through the gap 108. Hence, the guide 100 can be lifted upwards until it reaches the upper attachment region 116, then pulled away from the drill bit 110.

In operation, the user places the guide 100 on a patient's skull according to appropriate features or landmarks on the patient's skull. Once in a desired position and orientation, the user advances the drill bit 110 (which is coupled to drill 130) through the guide passage 106 of the guide 100. The drill bit 110 is further advanced until the drill head 112 touches the skin of the patient's skull and the drill stop 114 is held in place by the sides of the guide passage 106. At this time, the desired angular position of the drill bit 110 and drill has been achieved.

Next, the user maintains the angular position of the drill bit 110 and drill while moving the guide 100 upwards over the drill bit 110, over the stop 114. Once the passage 106 has move completely over the stop 114 and is positioned over the narrow region 116, the guide 100 is pulled off of the drill bit 110 such that the gap 108 in passage 106 moves over the narrow region 116. Hence, the guide 100 is completely removed from the drill bit 110 without angular or positional movement of the drill bit 110, allowing the user to better visualize the drilling process.

In an alternative embodiment, the gap 108 may be larger than the diameter of the widest portion of the drill bit 110 a latching door member that allows the user to selectively open the gap 108 and remove the guide 100 from the drill bit 110.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A system for creating an aperture in a skull comprising:
a guide body;
a plurality of support members disposed on an underside of said guide body;
a guide passage extending through said guide body and having a longitudinal gap along a side of said guide passage;
a drill bit having a first region and a second region;
wherein a diameter of said first region is larger than said longitudinal opening and wherein a diameter of said second region is smaller than said longitudinal opening.

2. The system of claim 1, wherein ends of said plurality of support members form a virtual plane and wherein said guide passage is oriented at a perpendicular angle to said virtual plane.

3. The system of claim 1, wherein said first region is a drill stop.

4. The system of claim 1, wherein said guide body comprises a first curved elongated member and a second curved elongated member and wherein said first curved, elongated member and said second curved elongated member form said guide passage.

5. The system of claim 4, wherein a first end of said first curved elongated member and a second end of said second curved elongated member form said longitudinal gap.

6. The system of claim 4, wherein said plurality of support members is three legs.

7. The system of claim 1, wherein said drill bit comprises a drill head configured for drilling, a drill stop positioned proximally of said drill head and a narrow drill bit region positioned proximally of said drill stop and configured to couple with a drill.

8. A system for creating a burr hole in a skull comprising:
a guide body;
at least three support members disposed on an underside of said guide body;
a first elongated member extending out from said guide body;
a second elongated member extending out from said guide body;
said first and second elongated members forming a guide passage extending through said guide body and having a longitudinal gap along a side of said guide passage;
a drill member having a first region and a second region;
wherein a diameter of said first region is larger than said longitudinal opening and wherein a diameter of said second region is smaller than said longitudinal opening.

9. The system of claim 8, wherein ends of said at least three support members align with a plane and wherein said guide passage is aligned perpendicularly to said plane.

10. The system of claim 9, wherein said first region is a drill stop.

11. The system of claim 10, wherein said drill bit comprises a drill head configured for drilling, a drill stop positioned proximally of said drill head and a narrow drill bit region positioned proximally of said drill stop and configured to couple with a drill.

12. The system of claim 11, wherein said guide body includes a third elongated member and a forth elongated member, each of which having a support member disposed at its end.

13. The system of claim 11, wherein said longitudinal opening is selectively opened by a latching door member.

14. A method of creating a burr hole in a skull comprising:
providing a guide member having a guide passage;
providing a drill having a drill bit;
placing said guide member on a skull;
aligning said drill bit relative to said skull by placing said drill bit into said guide passage;
lifting said guide member away from said skull; and,
pulling said guide member off of said drill bit through a longitudinal gap along a side of said guide passage.

15. The method of claim 14, wherein said lifting said guide member away from said skull further comprises lifting said guide member from a first region of said drill bit to a second region of said drill bit, said second region having a smaller diameter than said first region.

16. The method of claim 15, wherein said pulling said guide member off of said drill bit further comprises moving said second region through said longitudinal gap.

17. The method of claim 16, wherein said aligning said drill bit further comprises aligning said drill bit perpendicular to a plane formed by at least three support legs of said guide member.
